(19) United States
(12) Patent Application Publication
(10) Pub. No.: US 2012/0028045 A1
(43) Pub. Date: Feb. 2, 2012

(54) Processes for the Preparation of Indiplon and Intermediates Thereof

(75) Inventors: Joseph Prabahar Koilpillai, Maharashtra (IN); Sanjay Anantha Kale, Navi Mumbai (IN); Laxmikant Madhukar Kelkar, Mumbai (IN); Sunil Sudhakar Zope, Maharashtra (IN); Mubeen Ahmed Khan, Maharastra (IN)

(73) Assignee: Glenmark Generics LTD, Mumbai, Maharastra (IN)

(21) Appl. No.: 13/260,247

(22) PCT Filed: Mar. 18, 2010
(86) PCT No.: PCT/IN2010/000161

§ 371 Date: Sep. 23, 2011

(30) Foreign Application Priority Data
Mar. 24, 2009 (IN) 689/MUM/2009

(51) Int. Cl. B32B 5/16; C07D 487/04

(52) U.S. Cl. 428/402; 544/281

(57) ABSTRACT

The present invention relates to processes for the preparation of indiplon and its polymorphic mixtures.

### PRIORITY

This application claims the benefit to Indian Provisional Application 689/MUM/2009, filed on Mar. 24, 2009, the contents of which is incorporated by reference herein.

### BACKGROUND OF THE INVENTION

1. Technical Field

The present invention relates to processes for the preparation of indiplon and intermediates thereof. The present invention also relates to polymorphic mixtures of indiplon and processes for the preparation thereof.

2. Description of the Related Art

Indiplon is a GABA-A receptor modulator, which has been filed for regulatory approval in the U.S. for treatment of primary, chronic insomnia in adult and elderly patients. Indiplon is chemically described as N-methyl-N-[3-[3-(thien-2-ylcarbonyl)pyrazolo[1,5-a]pyrimidin-7-yl]phenyl]acetamide and is represented by structural formula (I).

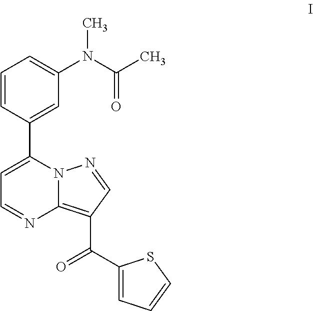

U.S. Pat. No. 4,521,422 (the '422 patent) describes pyrazolopyrimidines derivatives, including indiplon and their pharmaceutically acceptable salts, a pharmaceutical composition and method of treatment, a process for the preparation of indiplon.

U.S. Pat. No. 6,399,621 (the '621 patent) discloses a process, as illustrated below, for the preparation of indiplon, which involves an N-alkylation of enaminone intermediate compound of formula IV, that is carried out by using iodomethane in the presence of sodium hydride to give the N-methyl enaminone intermediate compound of formula III followed by condensation with aminopyrazole intermediate compound of formula II using glacial acetic acid at reflux conditions to afford indiplon of formula I.

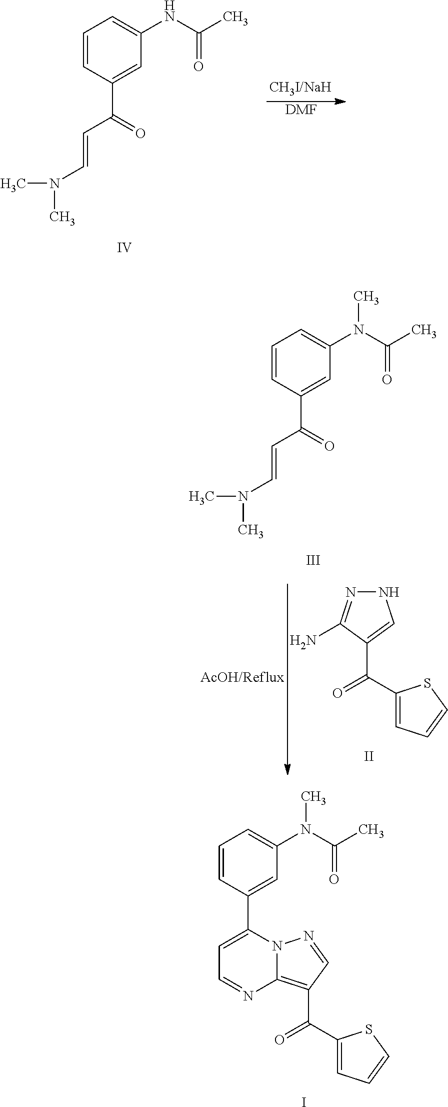

U.S. Pat. No. 6,472,528 (the '528 patent) describes a process for the preparation of indiplon comprising an N-alkylation of an enaminone intermediate compound of formula IV, that is carried out with the use of dimethyl sulfate, a phase transfer catalyst (tetrabutyl ammonium sulfate) in the presence of benzoflouride and dichloromethane as solvents at a temperature below 40° C., to give the N-methylenaminone intermediate compound of formula III, as illustrated below; then followed by condensation with aminopyrazole intermediate compound of formula II using glacial acetic acid at reflux conditions to afford indiplon of formula I.

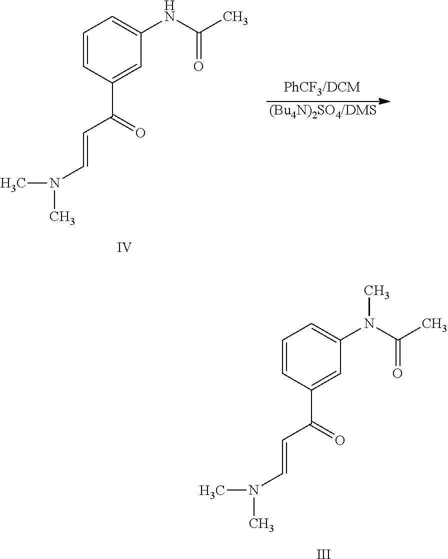

U.S. Pat. No. 6,348,221 (the '221 patent) discloses that indiplon obtained according to U.S. Pat. No. 4,521,422 (example 1), exist as a mixture of polymorphic Form I and Form II. Neither patent teaches nor discloses the preparation of the polymorphic mixture or the ratio therewith.

U.S. Pat. Nos. 6,348,221 and 6,544,999 disclose polymorphic Form I and Form II of indiplon. U.S. Pat. No. 6,903,106 discloses polymorphic Form III of indiplon.

In the aforementioned processes previously discussed, the N-alkylation of the enaminone intermediate compound (IV) engages differing methodologies; where one process uses sodium hydride as a base and alternately, another process uses a phase transfer catalyst. The similarity of these processes affords the N-methylenaminone compound of formula III to be produced in yields of less than about 60% and purity levels of less than about 90%, which translates to a reduction in the overall yield of indiplon (I).

Further, the usage of either sodium hydride as a base or phase transfer catalysts is disadvantageous, which stems from moisture sensitivity, safety concerns, storage issues, required quenching after use, which subsequently negates the suitability and feasibility of the process on an industrial scale.

The '621 and '528 patents disclose the recovery of the target indiplon product from the reaction mixture using water and glacial acetic acid. However, neither the '621 patent or the '528 patent discloses the formation or presence of by-products; and their removal or separation from the target product, should they be formed.

Potentially in acidic conditions the isomeric regioisomer impurity of indiplon may be formed in an amount of not less than 5%. The levels of impurity would make the product unacceptable to market. Additionally, said product to be marketable would require multiple purifications steps, thus rendering the process economically not feasible.

Moreover, for a new drug product to gain marketing approval, manufacturers are mandated to submit to the regulatory authorities, evidence to show that the product is acceptable for human administration. Such a submission must include, among other things, analytical data to show the impurity profile of the product to demonstrate that the impurities are absent, or are present only at a negligible amount.

Further, the U.S. Food and Drug Administration's Center for Drug Evaluation and Research (CDER) has promulgated guidelines recommending that new drug and generic drug applicants identify organic impurities of 0.1% or greater in the active ingredient. Unless an impurity is a human metabolite, has been tested for safety, or was present in a composition that was shown to be safe in clinical trials, the CDER further recommends that the drug applicant reduce the amount of the impurity in the active ingredient to below 0.1%. Thus, there is a need to isolate impurities in drug substances so that their pharmacology and toxicology can be studied.

In light of the evolving and more rigorous requirements demanded of drug manufacturers and the prevailing disadvantages present with the prior art, there is a need for an improved process for the preparation of indiplon and its intermediates, which circumvents the usage of potentially hazardous chemicals, the likely formation of isomeric and other process-related impurities; while ensuring a target indiplon product with optimum yield and purity.

The processes, herein described, for the preparation of indiplon and intermediates are simple, cost effective, eco-friendly and well suited on industrial scale.

### SUMMARY OF THE INVENTION

The present invention relates to processes for the preparation of indiplon and its polymorphic mixtures The present invention provides indiplon, having less than about 0.4% area of regioisomer impurity, as measured by HPLC.

The present invention provides indiplon, prepared by the processes herein described, having a purity of at least about 99.0% as measured by HPLC.

The present invention provides indiplon, prepared by the processes herein described, having a D₅₀ and D₉₀ particle size of less than about 50 microns.

The present invention provides indiplon, prepared by the processes herein described, having a D₅₀ and D₉₀ particle size of less than about 10 microns.

The present invention provides indiplon, prepared by the processes herein described, having no more than about 5000 ppm of acetone and ethanol, no more than about 3000 ppm of methanol, no more than about 1000 ppm of N,N-dimethylformamide, no more than about 600 ppm of dichloromethane, and/or no more than about 400 ppm of acetonitrile.

The present invention provides indiplon, prepared by the processes herein described, having a specific surface area of from about 1 m²/g to about 15 m²/g as measured by Brunauer-Emmett-Teller (B.E.T)

The present invention provides a process for preparing indiplon of formula I.

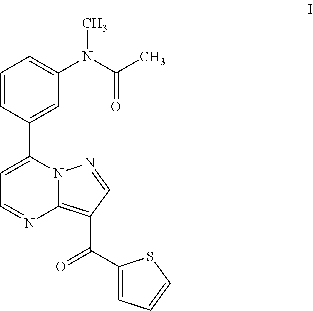

comprising:
a) reacting a compound N-[3-[3-(dimethylamino)-1-oxo-2-propenyl]-phenyl]-acetamide of formula IV or salt thereof

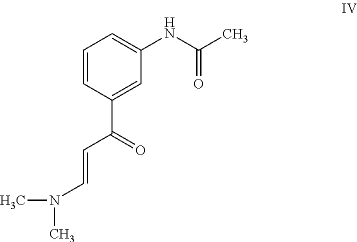

with a base, capable of producing hydroxide ions and methylating agent in the presence of an organic solvent to form the compound N-[3-[3-(dimethylamino)-1-oxo-2-propenyl]-phenyl]-N-methylacetamide of formula III or a salt thereof

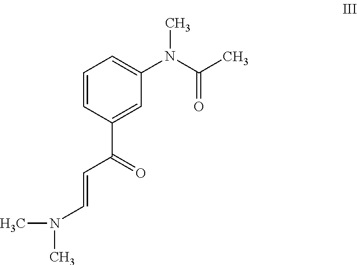

b) reacting the compound of formula III with the compound (3-amino-1H-pyrazol-4-yl)-2-thienylmethanone of formula II or a salt thereof

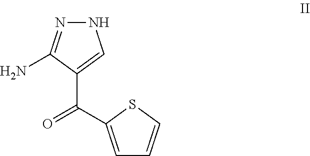

in the presence of an acid with a pka of below about 4 and an organic solvent.

The present invention provides a process for purifying indiplon comprising:

a) providing a solution of a indiplon, comprising a regioisomer impurity, in a solvent or a mixture of solvents or their aqueous mixtures and
b) precipitating the solid from the solution, and
c) recovering the solid to obtain indiplon substantially free of regioisomer.

The present invention provides a polymorphic mixture, comprising at least about 5 weight %, based on the total weight of the mixture, of polymorph Form I or polymorph Form II of indiplon, with remaining amount of the mixture being the other polymorph form of indiplon.

The present invention provides a polymorphic mixture, comprising about 25 weight % to about 90 weight % of polymorph Form I of indiplon and about 75 weight % to about 10 weight % of polymorph Form II of indiplon.

The present invention provides a polymorphic mixture, comprising about 40% of polymorph Form I of indiplon and about 60% of polymorph Form II of indiplon.

The present invention provides a polymorphic mixture, comprising about 70% of polymorph Form I of indiplon and about 30% of polymorph Form II of indiplon.

The present invention provides a polymorphic mixture, comprising about 75% of polymorph Form I of indiplon and about 25% of polymorph Form II of indiplon.

The present invention provides a process for the preparation of a mixture of polymorphic Form I and II of indiplon, comprising:

a) providing a solution of a indiplon, in a solvent or a mixture of solvents or their aqueous mixtures,
b) precipitating the solid from the solution, and
c) isolating the polymorphic mixture

The present invention provides a polymorphic mixture of indiplon, prepared by the processes herein described, having a D₅₀ and D₉₀ particle size of less than about 50 microns.

The present invention provides a polymorphic mixture of indiplon, prepared by the processes herein described, having a D₅₀ and D₉₀ particle size of less than about 10 microns.

The present invention provides indiplon, prepared by the processes herein described, having no more than about 5000 ppm of acetone and ethanol, no more than about 3000 ppm of methanol, no more than about 1000 ppm of N,N-dimethylformamide, no more than about 600 ppm of dichloromethane, and/or no more than about 400 ppm of acetonitrile.

The present invention provides a pharmaceutical composition comprising indiplon obtained by the processes herein described, and at least a pharmaceutically acceptable carrier.

### BRIEF DESCRIPTION OF DRAWING

FIG. 1. Scanning Electron Micrograph (SEM) of indiplon crystal particles obtained by the process of present invention.

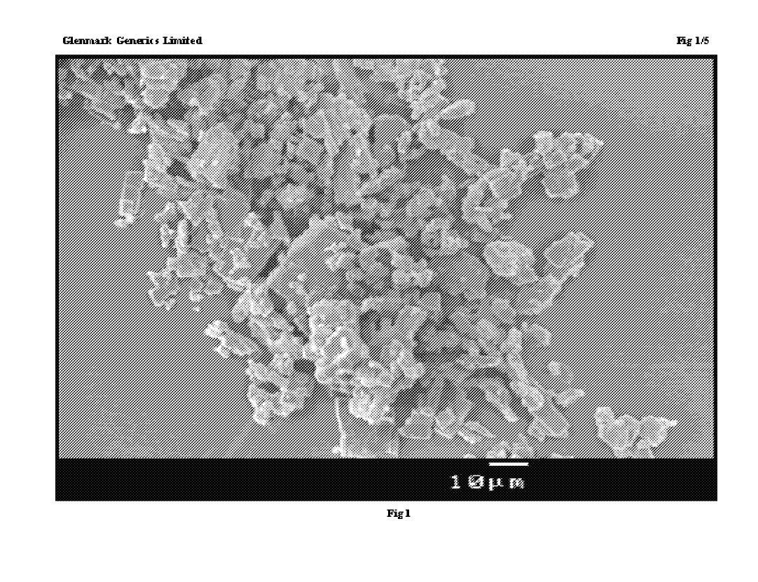

FIG. 2. Differential Scanning calorimetry (DSC) thermogram of polymorphic mixture Form I and Form II of indiplon prepared by example 11.

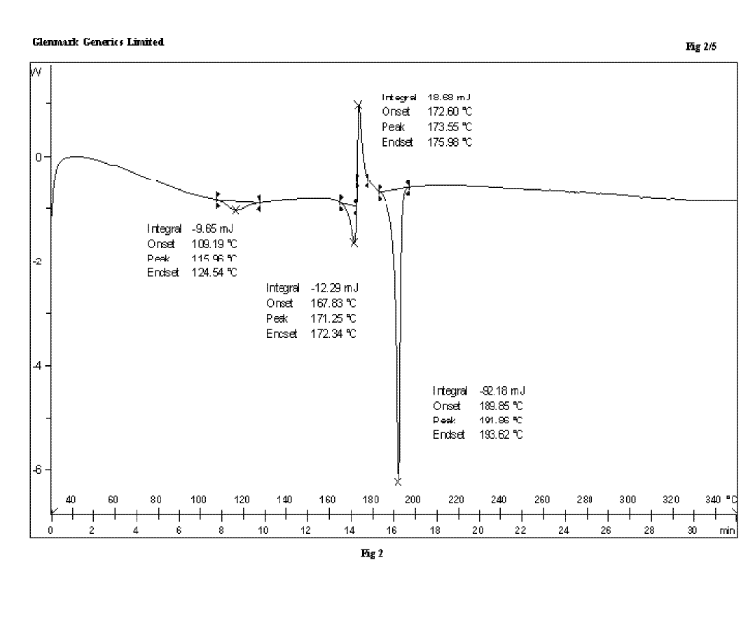

FIG. 3. X-ray powder diffraction (XRPD) spectrum of polymorphic mixture Form I and Form II of indiplon prepared by example 11.

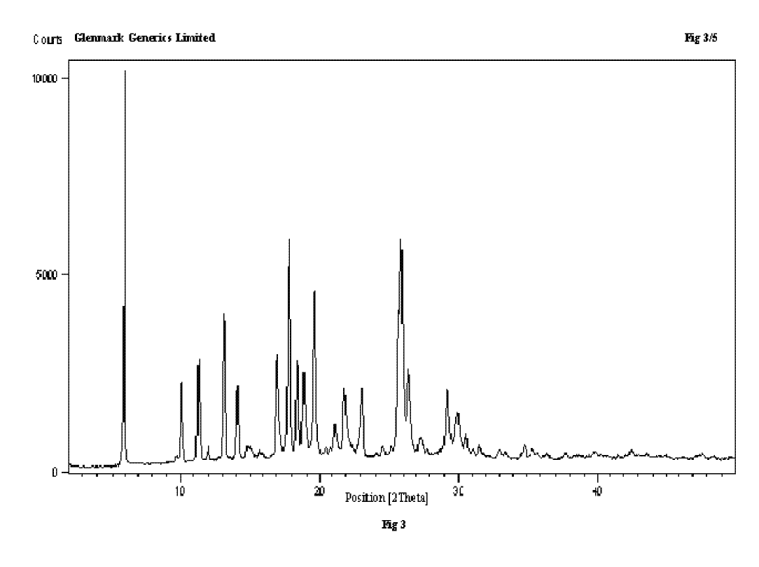

FIG. 4. Differential Scanning Calorimetry (DSC) thermogram of polymorphic mixture Form I and Form II of indiplon prepared by example 12.

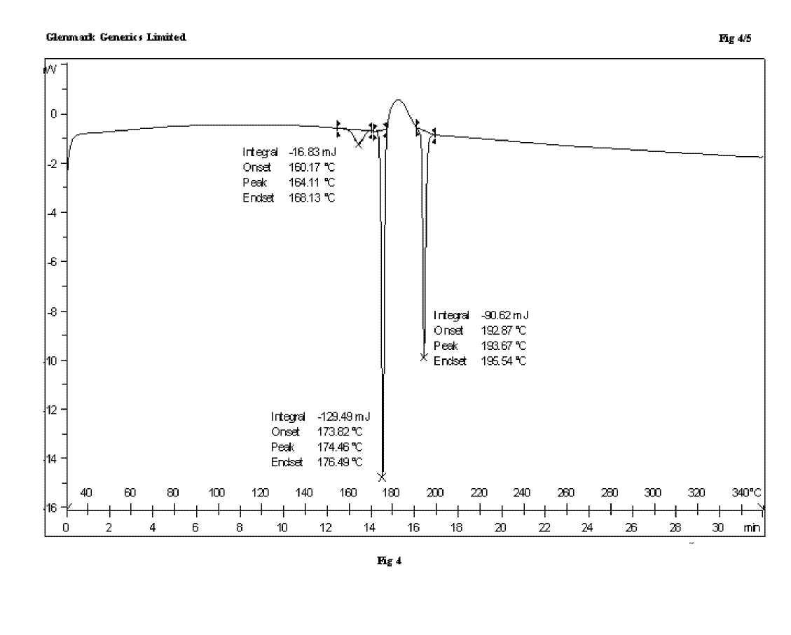

FIG. 5. Differential Scanning Calorimetry (DSC) thermogram of polymorphic mixture Form I and Form II of indiplon prepared by example 14.

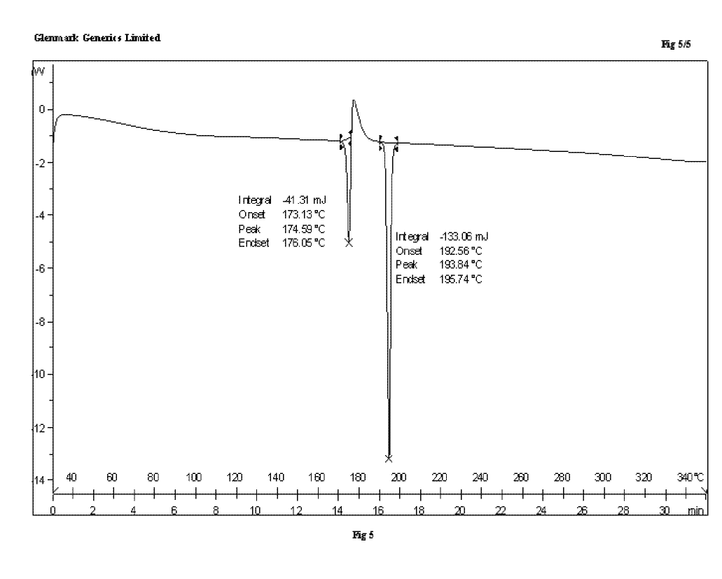

### DETAILED DESCRIPTION OF THE INVENTION

The present invention is directed to processes for the synthesis of indiplon and its polymorphic mixtures.

Present health care reforms and legislation lead to evolving and increasingly rigorous requirements demanded of drug manufacturers. Subsequent therefrom and coupled with prevailing disadvantages, which may be present with the prior art processes, paves opportunities for improved processes for the preparation of indiplon and its intermediates, which could circumvent the formation of process related impurities, while ensuring a target indiplon product with optimum yield and purity.

The present invention provides a cost effective industrial process for the preparation of indiplon or intermediates thereof.

In one embodiment, the present invention provides indiplon, having less than about 0.40% area of regioisomer impurity, as measured by high performance liquid chromatography (HPLC).

The present invention provides indiplon, prepared by the processes herein described, having a purity of at least about 99.0% as measured by HPLC.

The present invention provides a process for preparing indiplon of formula I,

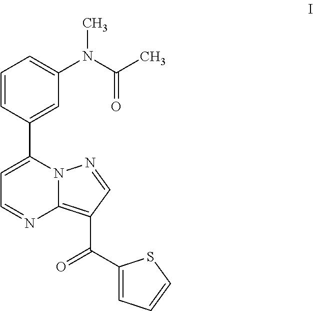

comprising:
a) reacting a compound N-[3-[3-(dimethylamino)-1-oxo-2-propenyl]-phenyl]-acetamide of formula IV or salt thereof

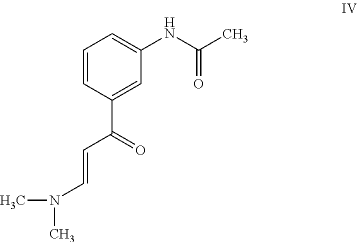

with a base capable of producing hydroxide ions and methylating agent in the presence of an organic solvent to form the compound N-[3-[3-(dimethylamino)-1-oxo-2-propenyl]-phenyl]-N-methylacetamide of formula III or a salt thereof

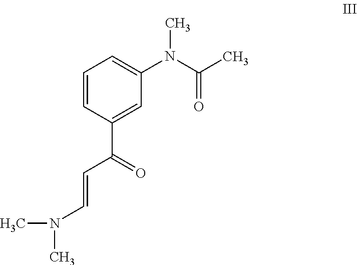

b) reacting the compound of formula III with the compound 3-amino-1H-pyrazol-4-yl)-2-thienylmethanone of formula II or a salt thereof

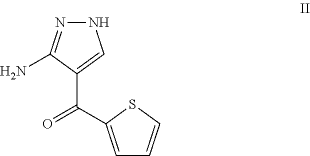

in the presence of an acid with a pka of below about 4 and an organic solvent.

In a) of the process directly described above, the base that can be used which is capable of providing hydroxide ions is selected from alkali metal hydroxides such as sodium hydroxide, potassium hydroxide, lithium hydroxide and the like; alkaline earth metal hydroxides such as magnesium hydroxide, calcium hydroxide and the like; ammonium hydroxide and mixtures thereof and their aqueous or alcoholic mixtures. The alkali and alkaline metal alkoxides, alkali and alkaline metal carbonates and bicarbonates are also contemplated, preferably potassium hydroxide.

The organic solvent include but are not limited to halogenated solvents such as dichloromethane, ethylene dichloride, chloroform and the like; esters solvent such as ethyl acetate, isopropyl acetate and the like; nitriles such as acetonitrile, propionitrile and the like; ethers such as tetrahydrofuran, 1,4-dioxane and the like; aprotic polar solvents such as N,N-dimethyl formamide, dimethyl sulfoxide, dimethyl acetamide, N-methyl-2-pyrrolidone, hexamethyl phosphoric triamide and mixtures thereof in various proportions without limitation. Preferably, N,N-dimethyl formamide (DMF).

Further, the methylating agents that can be used include, but are not limited to, methyl iodide, dimethyl sulphate and the like. Preferably, methyl iodide.

The temperatures for carrying out the reaction in a) can be from about 25° C. to about 40° C. Preferably, from about 25° C. to about 30° C.

The reaction time for the completion of reaction can be from about 30 minutes to about 5 hours. Preferably, about 30 minutes.

The amount of base employed in a) is from about an equimolar amount to about 5 times the equimolar amount with respect to the starting material of formula IV. Preferably an equimolar amount.

Optionally, when an excess base, which may be either an aqueous or an alcoholic mixture is employed, this, then, may additionally serve as the solvent.

The acid that can be used in b) above in the reaction of compound of formula III and compound of formula II include, but are not limited to acids having the pka of below about 4 such as hydrochloric acid, hydrobromic acid, hydroiodic acid, nitric acid, sulfuric acid, phosphoric acid, citric acid, formic acid, oxalic acid, maleic acid, succininc acid, benzoic acid, ascorbic acid, paratoluene sulfonic acid, methane sulfonic acid, and the like; and their aqueous mixtures thereof. Preferably, phosphoric acid or maleic acid or hydrochloric acid is being used.

The solvent in b) that can used include, but not limited to, a water miscible alcohol such as methanol, ethanol, isopropanol, n-butanol and the like; ketone such as acetone, methyl ethyl ketone, methyl isobutyl ketone and the like; nitrile such as acetonitrile, propionitrile and the like; and mixtures thereof in various proportions without limitation. Preferably, methanol or ethanol is used.

The temperatures for carrying out the reaction in b) can be from about 25° C. to about 50° C. Preferably from about 25° C. to about 35° C. More preferably from about 25° C. to 30° C.

The time required for the completion of reaction in b) can be from about 30 minutes to about 15 hours. Preferably from about 5 to 10 hours.

Typically, the molar amount of compound of formula II may be about 1 to about 2 times the molar amount of the compound of formula III. Preferably about 1 molar equivalent. While the molar equivalents of acid used may be about 1 to about 10 times the molar amount of the compound of formula III. Preferably, about 5 molar equivalents.

Optionally the reaction in b) is carried out in the absence of solvents, i.e., in neat conditions by employing an excess of aqueous acid.

Optionally the reaction in b) is carried without isolation of intermediates, i.e., can be carried out by one pot synthesis.

After completion of the reaction, the desired compounds of either or both formula III and formula I can be obtained from the reaction mixture by conventional means known to one of skilled in the art. Should the target compounds be produced immediately in the form of crystals, these can be optionally separated by filtration. Alternatively, a suitable recovery procedure optionally comprises: adding water; neutralizing the mixture, if necessary; extracting the mixture with a water-immiscible organic solvent; drying the extract; and distilling the solvent off. The product thus obtained can be, optionally further purified by conventional means, such as recrystallization or chromatographic separation techniques, for example preparative thin layer chromatography or column chromatography, notably column chromatography. Preferably by recrystallization.

The compounds of formulae IV and II can be prepared according to the methods described in U.S. Pat. Nos. 6,399,521 and 7,034,154, which are incorporated herein by reference, in their entirety.

The present invention, the processes are optionally carried out in situ; or by one pot synthesis.

The present invention, a compound of formulae IV or I is optionally purified by re-crystallization using a solvent or mixture of solvents.

The present invention, a compound of formulae IV or I is purified optionally by converting into a pharmaceutically acceptable salt.

In an embodiment, the present invention provides a process for purifying indiplon comprising:

a) providing a solution of a indiplon comprising regioisomer in a solvent or a mixture of solvents or their aqueous mixtures, and
b) precipitating the solid from the solution, and
c) recovering the solid to obtain indiplon substantially free of regioisomer.

The solvents that can be used in a) of the process directly described above, for the dissolution of indiplon is selected from a C₁-C₅ alcohol, a C₂-C₉ ester, a C₃-C₉ ketone, a C₃-C₅ carbonate, nitriles, ethers, hydrocarbon solvents and halogenated derivatives thereof, acetic acid, dimethylformamide (DMF), dimethylacetamide (DMAC), N-methylpyrrolidine, formamide, N-methylacetamide, N-methylformamide, dimethylsulfoxide (DMSO), ethylformate, sulfonate, N,N-dimethylpropionamide, nitromethane, nitrobenzene, and hexamethylphosphoramide, and mixtures thereof and mixtures of said organic solvents and water. Preferably acetone, acetonitrile, propionitrile, hexane, methanol, ethanol, isopropanol, diethyl ether, ethyl acetate, isobutyl acetate, dichloromethane, tetrahydrofuran, dimethyl formamide, dimethylsulfoxide, nitromethane and mixtures thereof and mixtures of said organic solvents and water. Preferably DMSO, nitromethane, isopropanol, isobutanol, methylethyl ketone, 1,4-dioxane, ethylene glycol, diethylene glycol dimethyl ether, hexane, dichloromethane and mixtures thereof and mixtures of said organic solvents and water. The C₁-C₅ are selected from methanol, ethanol, n-propanol, isopropanol, n-butanol, isobutanol, 2-butanol and the like; C₂-C₉ ester are selected from methyl acetate, ethyl acetate, isopropyl acetate, isobutyl acetate, n-butyl acetate, t-butyl acetate and the like; C₃-C₉ ketone are selected from acetone, 2-butanone, methylethyl ketone, ethylmethyl ketone, isopropylmethyl ketone, methyl isobutyl ketone and the like; C₃-C₅ carbonate are selected from dimethyl carbonate, diethyl carbonate and the like; nitriles are selected from acetonitrile, propionitrile and the like.

The ethers are selected from diethyl ether, dimethyl ether, dimethoxymethane, dimethoxypropane, isopropyl ether, di-isopropyl ether, methyl t-butyl ether, tetrahydrofuran (THF), dioxane, furan, ethylene glycol dimethyl ether, ethylene glycol diethyl ether, diethylene glycol dimethyl ether, diethylene glycol diethyl ether, anisole and the like. Hydrocarbon solvents and halogenated derivatives thereof, are selected from pentane, hexane, heptane, cyclohexane, petroleum ether, toluene, benzene, cycloheptane, methylcyclohexane, ethylbenzene, m-, o-, or p-xylene, octane, indane, nonane, dichloromethane (MDC), chloroform, carbon tetrachloride, 1,2-dichloroethane and the like.

The temperature for dissolution can range from about 25° C. to about 100° C. or reflux temperatures of the solvents used. Preferably at about 30° C.

The time period for dissolution can be range from about 30 minutes to about 5 hours. Preferably, 1 hour.

The solution obtained is optionally filtered through celite or diatomaceous earth to separate the extraneous matter present or formed in the solution by using conventional filtration technique known in the art.

The precipitation of solid in b) above is achieved but not limited to evaporation, cooling, drying and the like. Preferably, by cooling.

The temperature range for precipitation of solid can be from about −10° C. to about 30° C. Preferably about 30° C.

The time period for complete precipitation of solid can range from about 30 minutes to about 5 hours. Preferably 1 hour.

The obtained indiplon of formula I can be dried can be from about 25° C. to about 75° C., preferably at 50° C. and at reduced pressure of about e.g. 5 to 20 mbar, for a period of about 1 to about 10 hours. Preferably 1 hour.

In another embodiment, the present invention provides a polymorphic mixture, comprising at least about 5 weight %, based on the total weight of the mixture, of polymorph Form I or polymorph Form II of indiplon, with remaining amount of the mixture being the other polymorph form of indiplon.

The present invention provides a polymorphic mixture, comprising at least about 10 weight %, based on the total weight of the mixture, of polymorph Form I or polymorph Form II of indiplon, with remaining amount of the mixture being the other polymorph form of indiplon.

The present invention provides a polymorphic mixture, comprising at least about 15 weight %, based on the total weight of the mixture, of polymorph Form I or polymorph Form II of indiplon, with remaining amount of the mixture being the other polymorph form of indiplon.

The present invention provides a polymorphic mixture, comprising at least about 20 weight %, based on the total weight of the mixture, of polymorph Form I or polymorph Form II of indiplon, with remaining amount of the mixture being the other polymorph form of indiplon.

The present invention provides a polymorphic mixture, comprising at least about 25 weight %, based on the total weight of the mixture, of polymorph Form I or polymorph Form II of indiplon, with remaining amount of the mixture being the other polymorph form of indiplon.

The present invention provides a polymorphic mixture, comprising about 25 weight % to about 90 weight % of polymorph Form I of indiplon and about 75 weight % to about 10 weight % of polymorph Form II of indiplon.

The present invention provides a polymorphic mixture, comprising about 80 weight % (±5%) of polymorph Form I of indiplon and about 20 weight % (±5%) of polymorph Form II of indiplon

The present invention provides a polymorphic mixture, comprising about 70 weight % (±5%) of polymorph Form I of indiplon and about 30 weight % (±5%) of polymorph Form II of indiplon

The present invention provides a polymorphic mixture, comprising about 60 weight % (±5%) of polymorph Form I of indiplon and about 40 weight % (±5%) of polymorph Form II of indiplon.

The present invention provides a polymorphic mixture, comprising about 50 weight % (±5%) of polymorph Form I of indiplon and about 50 weight % (±5%) of polymorph Form II of indiplon.

The present invention provides a polymorphic mixture, comprising about 40 weight % (±5%) of polymorph Form I of indiplon and about 60 weight % (±5%) of polymorph Form II of indiplon.

The present invention provides a polymorphic mixture, comprising about 30 weight % (±5%) of polymorph Form I of indiplon and about 70 weight % (±5%) of polymorph Form II of indiplon.

The present invention provides a polymorphic mixture, comprising about 40% of polymorph Form I of indiplon and about 60% of polymorph Form II of indiplon.

The present invention provides a polymorphic mixture, comprising about 70% of polymorph Form I of indiplon and about 30% of polymorph Form II of indiplon.

The present invention provides a polymorphic mixture, comprising about 75% of polymorph Form I of indiplon and about 25% of polymorph Form II of indiplon.

In yet another embodiment, the present invention provides a process for the preparation of a mixture polymorphic Form I and II of indiplon, comprising:

a) providing a solution of a indiplon, in a solvent or a mixture of solvents or their aqueous mixtures,
b) precipitating the solid from the solution, and
c) isolating the polymorphic mixture

The solvent that can be used include but are not limited to C₁-C₅ alcohol, a C₂-C₉ ester, a C₃-C₉ ketone, a C₃-C₅ carbonate, nitriles, ethers, hydrocarbon solvents and halogenated derivatives thereof, acetic acid, dimethylformamide (DMF), dimethylacetamide (DMAC), N-methylpyrrolidine, formamide, N-methylacetamide, N-methylformamide, dimethylsulfoxide (DMSO), ethylformate, sulfonate, N,N-dimethylpropionamide, nitromethane, nitrobenzene, and hexamethylphosphoramide, and mixtures thereof and mixtures of said organic solvents and water. Preferably DMSO, nitromethane, isopropanol, isobutanol, methylethyl ketone, 1,4-dioxane, ethylene glycol, diethylene glycol dimethyl ether, tetrahydrofuran, hexane, dichloromethane and mixtures thereof and mixtures of said organic solvents and water. The C₁-C₅ alcohol include methanol, ethanol, n-propanol, isopropanol, n-butanol, isobutanol, 2-butanol and the like; C₂-C₉ ester include methyl acetate, ethyl acetate, isopropyl acetate, isobutyl acetate, n-butyl acetate, t-butyl acetate and the like; C₃-C₉ ketone acetone, 2-butanone, methylethyl ketone, ethylmethyl ketone, isopropylmethyl ketone, methyl isobutyl ketone and the like; C₃-C₅ carbonate includes dimethyl carbonate, diethyl carbonate and the like; nitriles such as acetonitrile, propionitrile and the like. The ethers include diethyl ether, dimethyl ether, dimethoxymethane, dimethoxypropane, isopropyl ether, di-isopropyl ether, methyl t-butyl ether, tetrahydrofuran (THF), dioxane, furan, ethylene glycol dimethyl ether, ethylene glycol diethyl ether, diethylene glycol dimethyl ether, diethylene glycol diethyl ether, anisole and the like. Hydrocarbon solvents and halogenated derivatives thereof, may include pentane, hexane, heptane, cyclohexane, petroleum ether, toluene, benzene, cycloheptane, methylcyclohexane, ethylbenzene, m-, o-, or p-xylene, octane, indane, nonane, dichloromethane (MDC), chloroform, carbon tetrachloride, 1,2-dichloroethane and the like.

If desired, pure indiplon obtained by the process of the present invention and having a purity of at least 99%, as determined by HPLC, can be further recrystallized from a solvent, preferably from methanol, ethanol, or a reaction medium of water and a co-solvent such as methanol, ethanol, acetonitrile and the like in order to produce a drug substance that complies with regulatory requirements.

The regioisomer of indiplon is represented by formula V

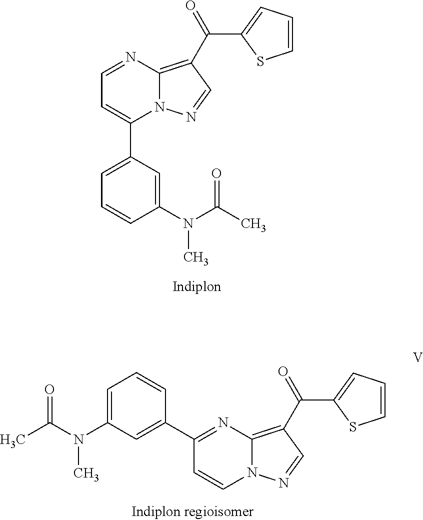

Formation of N-(3-[3-[2-thienylcarbonyl]-pyrazol-[1,5-a]pyrimidin-5-yl]phenyl)N-methylacetamide of formula V, regioisomer of indiplon, has been identified as a main impurity in the synthesis of indiplon starting from 3-amino-1H-pyrazol-4-yl)-2-thienylmethanone and N-[3-[3-(dimethylamino)-1-oxo-2-propenyl]-phenyl]-N-methylacetamide. The amount of this impurity has been found to be strongly dependent on the reaction conditions.

Apart from the regioisomer described above, the following process related impurities may be formed which are NMT 0.5% area by HPLC in total in the final product indiplon.

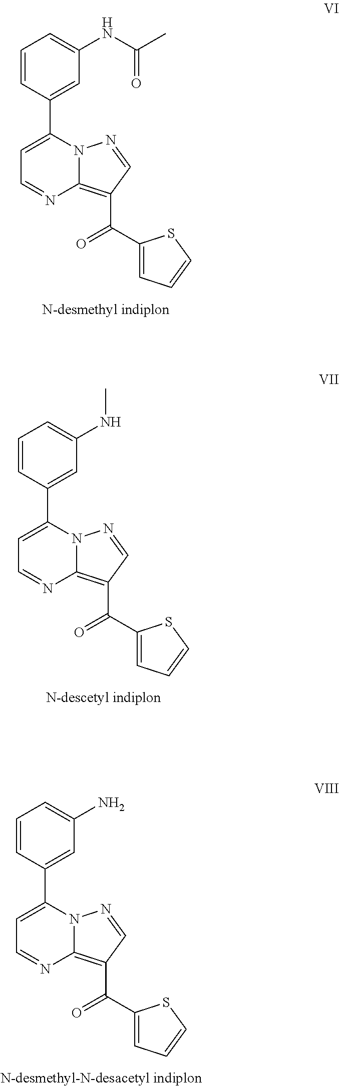

Indiplon obtained by the process of present invention has the purity of at least about 99.0 area % as measured by HPLC.

In another embodiment, the present invention provides indiplon of formula I, characterized by HPLC having a purity of at least about 99.0% and containing total impurities of about NMT 1.0%.

In yet another embodiment, indiplon obtained by the process described herein has a residual organic solvent content of less than the amount recommended for pharmaceutical products, as set forth for example in ICH guidelines and U.S. pharmacopoeia; i.e., less than about 600 ppm of dichloromethane, less than about 1000 ppm of N,N-dimethyl formamide, less than about 5000 ppm of ethanol, less than about 3000 ppm of methanol, less than about 5000 ppm of acetone and less than about 400 ppm of acetonitrile.

Crystal particles of indiplon used herein has the surface area of about 1 to about 15 m²/g as measured by B.ET (Brunauer-Emmett-Teller). Preferably from about 1 to about 5 m²/gm. The mean particle size of about 5 to about 50 μm. Preferably of about 5 to about 20 μm.

| S. No. | Test | Results |
|---|---|---|
| 1 | Description | Yellow coloured powder |
| 2 | Sulphated ash | 0.05% |
| 3 | Related Impurity by | * Impurity A: Not detected |
| | HPLC | **Impurity B: Below detection limit. Single |
| | | maximum impurity: 0.86% |
| | | Total impurities: 0.96% |
| 4 | TGA | 0.2725% sample weight lost up to 100° C. |
| 5 | Particle size | d(0.1) 4.949 μm |
| | Distribution | d(0.5) 11.641 μm |
| | | d(0.9) 24.325 μm |
| 6 | Bulk Density | As such = 0.24 g/ml |
| | | After tapping (750) = 0.47 g/ml as per USP |
| | | method I |
| 6 | SEM (Scanning | Plate shape crystals |
| | electron microscope) | |
| 7 | Surface area by BET | 1.41 m²/gm |

Impurity A: (3-Amino-1H-pyrazole-4-yl)-2-thienylmethanone.
Impurity B: N-[3-(3-Dimethylamino)-1-oxo-2-propenyl]phenyl]-N-methylacetamide.

As used herein, the term “μm” refers to “micrometer” which is 1×10⁻⁶ meter.

As used herein, “crystalline particles” means any combination of single crystals, aggregates and agglomerates.

As used herein “Particle Size Distribution (P.S.D.)” means the cumulative volume size distribution of equivalent spherical diameters as determined by laser diffraction at 1 bar dispersive pressure in Sympatec Helos equipment.

“Mean particle size distribution, i.e., d (0.5)” correspondingly, means the median of said particle size distribution.

Specific surface area is defined in units of square meters per gram (m²/g). It is usually measured by nitrogen absorption analysis. In this analysis, nitrogen is absorbed on the surface of the substance. The amount of the absorbed nitrogen (as measured during the absorption or the subsequent desorption process) is related to the surface area via a formula known as the B. ET. formula.

Specific surface area of an active pharmaceutical ingredient may be affected by various factors. There is an inverse relationship between specific surface area and particle size distribution The available surface area for drug dissolution correlates to the rate of dissolution and solubility where a greater surface area enhances the solubility of a drug and enhances the rate of dissolution of a drug, which, in effect may improve the drug's bioavailability and potentially its toxicity profiles. The lack of solubility of indiplon creates a problem since the bioavailability of a water insoluble active ingredient is usually poor. Thus there is a need in the art to prepare active pharmaceutical ingredients such as indiplon with a high surface area to obtain formulations with greater bioavailability, and to compensate for any loss of surface area before formulation.

The particle size can be determined by such techniques as, for example, Malvern light scattering, a laser light scattering technique, etc., while herein, used Malvern Mastersizer 2000. It is noted the notation Dₓ means that X % of the particles have a diameter less than a specified diameter D. The particle sizes of the Indiplon can be obtained by, for example, any milling, grinding, micronizing or other particle size reduction method known in the art to bring the solid state indiplon any of the foregoing desired particle size range.

The present invention provides a pharmaceutical composition comprising indiplon obtained by the process of present invention and suitable pharmaceutical carriers. The pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like. The pharmaceutical compositions comprising indiplon or its pharmaceutically acceptable salts, obtained by the process disclosed herein, and suitable pharmaceutical carriers also may be administered as suppositories, ophthalmic ointments and suspensions, and parenteral suspensions, where the most preferred route of administration is oral.

Capsule dosages will contain the indiplon or its pharmaceutically acceptable salts which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethyl-cellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The process for the preparation of indiplon or its pharmaceutically acceptable salts of hydrochloride of the present invention are simple, eco-friendly and easily scaleable.

The following examples are provided to enable one skilled in the art to practice the invention and are merely illustrative of the invention. The examples should not be read as limiting the scope of the invention as defined in the features and advantages.

### EXAMPLES

### Example 1

### Preparation of N-[3-[3-(dimethylamino)-1-oxo-2-propenyl]-phenyl]-N-methylacetamide (Formula III)

To a suspension of N-[3-[3-(dimethylamino)-1-oxo-2-propenyl]-phenyl]-acetamide (50 gm) in anhydrous dimethylformamide (258 ml) under nitrogen in an ice-bath was added sodium hydride (60% suspension in mineral oil)-(10.76 gm) and within about 15 minutes, it was observed that gas formation ceased. To the above reaction mixture is added a solution of methyl iodide (32.17 gm). The reaction mixture is stirred overnight and allowed to warm to room temperature. The reaction mass is then triturated with n-hexane (3×340 ml) which is discarded. The reaction mixture is then poured in ice water and extracted with methylene dichloride (3×45 ml). The organic layer is dried on sodium sulfate and evaporated to give a solid, which is triturated with a solution of n-hexane-ethyl acetate (1:1,453 ml) to give a solid product (22 gm) (Purity, ˜90%, by thin-layer chromatography, TLC)

### Example 2

### Preparation of N-[3-[3-(Dimethylamino)-1-oxo-2-propenyl]-phenyl]-n-methylacetamide (Formula III)

To a suspension of N-[3-[3-(dimethylamino)-1-oxo-2-propenyl]-phenyl]-acetamide (230 gm) in dimethylformamide (1150 ml) was added powdered potassium hydroxide (110.90 gm) and the reaction mixture was stirred at about 25° C.-30° C. for about 30 min. The reaction mass was cooled to about 0° C.-5° C. and methyl iodide (154.67 gm) was added in about 30 min at about the same temperature. After complete addition, the temperature was allowed to rise to about 20° C.-25° C., and while stirring, the reaction mass at about the same temperature until the reaction is completed (˜3 hrs) as monitored via thin-layer chromatography (TLC). To the reaction mass was added methylene chloride (500 ml) and water (500 ml), separated the organic layer and the aqueous layer was extracted with 2×500 ml methylene chloride. The combined organic layers were washed with 3×500 ml water, the subsequent organic layer was dried over sodium sulfate and concentrated to dryness to give a yellow colored solid, which was triturated with n-hexane (700 ml), filtered and dried at about 50° C. until a constant weight to give a solid product (190 gm) (Purity, ˜>95%, by TLC).

### Example 3

### Preparation of Indiplon (Formula I)

A mixture of (3-Amino-1H-pyrazol-4-yl)-2-thienylmethanone (10 gm) and N-[3-(dimethylamino)-1-oxo-2-propenyl]-phenyl]-N-methyl acetamide (12.5 gm) in glacial acetic acid (180 ml) was refluxed for about 6 hrs, and the progress of the reaction was monitored by TLC. The reaction mass was concentrated to obtain a residue, which was treated with methylene chloride (45 ml) and triturated with n-hexane (180 ml). The precipitated product was filtered, washed with a mixture of methylene chloride and n-hexane (1:1, 90 ml) and dried under vacuum to obtain 18 gm of the title compound. Purity by HPLC: 94.88%.

### Example 4

### Preparation of Indiplon (Formula I)

A mixture of (3-Amino-1H-pyrazol-4-yl)-2-thienylmethanone (110 gm) and N-[3-(dimethylamino)-1-oxo-2-propenyl]-phenyl]-N-methyl acetamide (140.22 gm) in glacial acetic acid (880 ml) was refluxed for about 6 hrs, and the progress of the reaction was monitored by TLC. The reaction mass was concentrated to obtain a residue, which was treated with acetone (440 ml) and stirred the precipitate for about 2 hrs at about 25-30°. The precipitated product was filtered, washed with acetone (110 ml) and dried in vacuum to obtain 175 gm of crude indiplon. Crude Indiplon (160 gm) was dissolved in acetone (6400 ml) at reflux temperature and DM water (3200 ml) was added in about 30 min. and cooled the reaction mass at about room temperature gradually and cooled at about 10-15° C., seeded with indiplon and stirred the precipitate for about 1 hr at about 10-15°. The precipitated product was filtered, washed with acetone (160 ml) and dried in vacuum to obtain 70 gm of the title compound. Crude Indiplon (35 gm) was dissolved in acetone (1750 ml) at the reflux temperature and DM water (875 ml) was added in about 30 min. and cooled the reaction mass at about room temperature gradually and cooled at about 10-15° C. and seeded with indiplon and stirred the precipitate for about 1 hr at about 10-15°. The precipitated product was filtered, washed with acetone (70 ml) and dried in vacuum to obtain 13 gm of the title compound. Regioisomer content is about 0.35%.

### Example 5

### Preparation of Indiplon Using Phosphoric Acid

N-[3-(Dimethylamino)-1-oxo-2-propenyl]-phenyl]-N-methylacetamide (2.5 gm, 0.010 mole) and (3-Amino-1H-pyrazol-4-yl)-2-thienylmethanone (2.0 gm, 0.010 mole) were dissolved in a mixture of water (30 ml), ethanol (15 ml) and phosphoric acid (0.79 gm, 0.0081 mol) and stirred for ˜10 hrs at room temperature, the progress of the reaction was monitored by TLC. After the completion of the reaction, the reaction mass was filtered, then washed with water (10 ml) and acetone (10 ml). The wet solid obtained was further dried at 50° C. under vacuum to give 2.52 gm of the title compound. Purity by HPLC: 99.14%.

### Example 6

### Preparation of Indiplon Using Aqueous Methanol and Hydrochloric Acid

N-[3-(Dimethylamino)-1-oxo-2-propenyl]-phenyl]-N-methyl acetamide (2.5 gm, 0.010 mol) and (3-Amino-1H-pyrazol-4-yl)-2-thienylmethanone (2.0 gm, 0.010 mol) were dissolved in a mixture of water (30 ml), methanol (15 ml) and ˜34% w/w, aq. hydrochloric acid (0.96 gm, 0.012 mol) and stirred for ˜10 hrs at room temperature, the progress of the reaction was monitored by TLC. After the completion of the reaction, the reaction mass was filtered, washed with water (10 ml) and acetone (10 ml). The wet solid obtained was further dried at 50° C. under vacuum to give 2.62 gm of the title compound. Purity by HPLC: 98.79%.

### Example 7

### Preparation of Indiplon Using Formic Acid

N-[3-(Dimethylamino)-1-oxo-2-propenyl]-phenyl]-N-methylacetamide (2.5 gm, 0.010 mol) and (3-Amino-1H-pyrazol-4-yl)-2-thienylmethanone (2.0 gm, 0.010 mole) were dissolved in formic acid (20 ml) and stirred at reflux temperature for ˜4 hrs, the progress of the reaction was monitored by TLC. After the completion of the reaction, the reaction mass was concentrated, the residue was triturated with acetone (20 ml) and filtered, washed with acetone (10 ml), the wet solid obtained was further dried at 45° C.-50° C. under vacuum to give 2.50 gm of the title compound. Purity by HPLC: 98.82%.

### Example 8

### Preparation of Indiplon Using Citric Acid

N-[3-(Dimethylamino)-1-oxo-2-propenyl]-phenyl]-N-methyl acetamide (5 gm, 0.020 mole) and (3-Amino-1H-pyrazol-4-yl)-2-thienylmethanone (4 gm, 0.020 mole) were dissolved in a mixture of water (54 ml), ethanol (31 ml) and citric acid (3.84 gm, 0.020 mol) and stirred for about 12 hrs at room temperature, and the progress of reaction was monitored by TLC. After completion of the reaction, the reaction mass was filtered, washed with a mixture of ethanol and water (1:1,24 ml). The wet solid obtained was further dried at about 50° C. under vacuum to give 5.30 gms of the title compound. Purity by HPLC: 99.10%.

### Example 9

### Preparation of Indiplon Using Maleic Acid

N-[3-(Dimethylamino)-1-oxo-2-propenyl]-phenyl]-N-methyl acetamide (5 gm, 0.020 mole) and (3-Amino-1H-pyrazol-4-yl)-2-thienylmethanone (4 gm, 0.020 mole) were dissolved in a mixture of water (54 ml), ethanol (3 ml) and maleic acid (2.40 gm, 0.020 mol) and stirred for about 12 hrs at room temperature, and the progress of the reaction was monitored by TLC. After completion of reaction, the reaction mass was filtered, washed with a mixture of ethanol and water (1:1, 24 ml). The wet solid obtained was further dried at about 50° C. under vacuum to give 5.40 gms of the title compound. Purity by HPLC: 99.24%.

### Example 10

### Preparation of Indiplon Using Oxalic Acid

N-[3-(Dimethylamino)-1-oxo-2-propenyl]-phenyl]-N-methyl acetamide (5 gm, 0.020 mole) and (3-Amino-1H-pyrazol-4-yl)-2-thienylmethanone (4 gm, 0.020 mole) were dissolved in a mixture of water (54 ml), ethanol (31 ml) and oxalic acid dihydrate (2.60 gm, 0.020 mol) and stirred for ˜12 hr at room temperature and the progress of the reaction was monitored by TLC. After completion of reaction, the reaction mass was filtered and washed with a mixture of ethanol and water (1:1, 24 ml). The wet solid obtained was further dried at 50° C. under vacuum to afford 5.10 gms of the title compound. Purity by HPLC: 98.62%.

### Example 11

### Preparation of Polymorphic Mixture of Form I and Form II of Indiplon

A mixture of (3-Amino-1H-pyrazol-4-yl)-2-thienylmethanone (12 gm) and N-[3-(dimethylamino)-1-oxo-2-propenyl]-phenyl]-N-methyl acetamide (15.29 gm) in glacial acetic acid (624 ml) was refluxed for about 6 hrs, and the progress of the reaction was monitored by TLC. The reaction mass was concentrated to obtain a residue, which was treated with methylene chloride (120 ml) and added saturated sodium bicarbonate solution and adjusted the pH to about 7.0 Stirred and separated organic layer and dried over sodium sulphate filtered and refluxed at the reflux temperature and added hexane (108 ml) at the reflux temperature, stirred and maintained reflux for about 15-20 min and cooled to about 25-30° C. and then cooled it to about 0° C. The precipitated product was filtered, dried in vacuum to obtain 16 gm of about 85% of Form I of indiplon and about 15% of Form II of indiplon. Purity by HPLC: 97.09%.

### Example 12

### Preparation of Polymorphic Mixture of Form I and Form II of Indiplon

Crude indiplon (4 gm) was dissolved in tetrahydrofuran (120 ml) at about the reflux temperature and filtered the solution and n-hexane (100 ml) was added, stirred the precipitate for about 30 min at about 80° C., gradually cooled the slurry mass at about 0-5° C. and stirred for about 1 hr at about the same temperature. The precipitated product was filtered, washed with n-hexane (4 ml) and dried in vacuum to obtain 3.5 gm of about 40% of Form I of indiplon and about 60% of Form II of indiplon.

### Example 13

### Preparation of Polymorphic Mixture of Form I and Form II of Indiplon

Crude indiplon (2 gm) was dissolved in methylene chloride (20 ml) at reflux temperature and was added n-hexane (6.0 ml) in about 10 min. and gradually cooled the reaction mass at about room temperature and thereafter cooled at about 0-5° C. and stirred for about 15 min. To the solution, 2.0 ml n-hexane was added and stirred the precipitate for about 15 min at about 0-5°. The precipitated product was filtered, washed with hexane (2 ml) and dried in vacuum to obtain 0.9 gm of about 75% of Form I of indiplon and about 25% of Form II of indiplon.

### Example 14

### Preparation of Polymorphic Mixture of Form I and Form II of Indiplon

Crude indiplon (2 gm) was dissolved in methylethylketone (100 ml) at about the reflux temperature and stirred for about 30 min at about 80° C., gradually cooled the reaction mass to about room temperature and stirred the precipitate for about 1 hr at about 25-30° C. The precipitated product was filtered, washed with methylethylketone (6 ml) and dried in vacuum to obtain 1.5 gm of about 70% of Form I of indiplon and about 30% of Form II of indiplon.

1. Indiplon having less than about 0.4% area of regioisomer impurity as measured by HPLC.

2. The compound of claim 1, having a D₅₀ and D₉₀ particle size of less than about 50 microns.

3. The compound of claim 2, having a D₅₀ and D₉₀ particle size of less than about 10 microns.

4. The compound of claim 1, further having a specific surface area of from about 1 m²/g to about 15 m²/g as measured by Brunauer-Emmett-Teller (B.E.T).

5. A process for the preparation of indiplon of formula I, 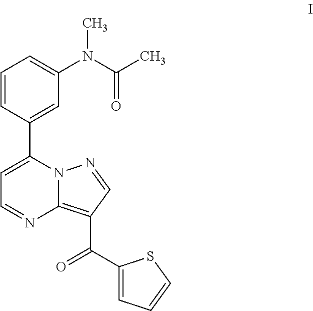
comprising:
a) reacting a compound N-[3-[3-(dimethylamino)-1-oxo-2-propenyl]-phenyl]-acetamide of formula IV or salt thereof 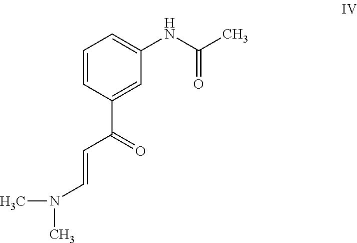
with a base capable of producing hydroxide ions and a methylating agent in the presence of an organic solvent to form the compound N-[3-[3-(dimethylamino)-1-oxo-2-propenyl]-phenyl]-N-methylacetamide of formula III or a salt thereof 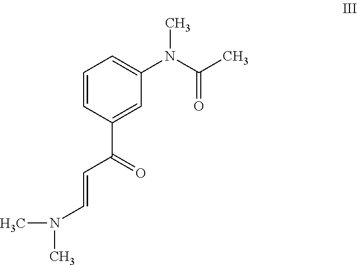
b) reacting the compound of formula III with the compound 3-amino-1H-pyrazol-4-yl)-2-thienylmethanone of formula II or a salt thereof 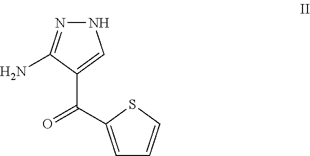
in the presence of an acid with a pKa of below about 4 in an organic solvent.

6. The process of claim 5, wherein the base capable of providing hydroxide ions is selected from the group comprising of alkali metal hydroxides such as sodium hydroxide, potassium hydroxide or lithium hydroxide, and alkaline earth metal hydroxides such as magnesium hydroxide, calcium hydroxide and mixtures thereof.

7. The process of claim 5, wherein the acid with a pKa of below about 4 is selected from hydrochloric acid, hydrobromic acid, hydroiodic acid, nitric acid, sulfuric acid, phosphoric acid, citric acid, formic acid, oxalic acid, maleic acid, succinic acid, benzoic acid, ascorbic acid, paratoluenesulfonic acid, methane sulfonic acid, and their aqueous mixtures thereof.

8. The process of claim 5, wherein the organic solvent used in the reaction of compound of formula IV with the base is a selected from halogenated solvents, dichloromethane, ethylene dichloride, chloroform, esters solvents ethyl acetate, isopropyl acetate, nitriles, acetonitrile, propionitrile, ethers tetrahydrofuran, 1,4-dioxane, aprotic polar solvents, N,N-dimethyl formamide, dimethyl sulfoxide, dimethylacetamide, N-methyl-2-pyrrolidone, hexamethyl phosphoric triamide and mixtures thereof.

9-19. (canceled)
